(19) United States
(12) Patent Application Publication
(10) Pub. No.: US 2018/0169000 A1
(43) Pub. Date: Jun. 21, 2018

(54) DYING AGENT USING NATURAL HENNA

(71) Applicant: EDP INC, Daejeon (KR)

(72) Inventor: Duheon PARK, Deajeon (KR)

(21) Appl. No.: 14/914,322

(22) PCT Filed: Sep. 7, 2015
(86) PCT No.: PCT/KR2015/009393

§ 371 Date: Feb. 25, 2016

(51) Int. Cl. A61K 8/9789; A61K 8/9728; A61Q 5/10

(52) U.S. Cl. CPC A61K 8/9789; A61K 8/9728; A61K 2800/85; A61K 2800/432; A61Q 5/10

(57) ABSTRACT

The present invention relates to a dying agent using natural henna, and more specifically, to a dying agent using natural henna with increased absorption of dye and nutrients and resultant shortened dying time and with minimized damage to hair or scalp upon dying.

According to the present invention, a dying agent using natural henna comprises a fermented mixture of 80 wt % to 99 wt % of natural henna and 1 wt % to 20 wt % of fermenting agent and a natural additive mixed with the fermented mixture.

This application is a 371 of international PCT Patent Application PCT/KR2015/009393 filed Sep. 7, 2015.

### TECHNICAL FIELD

The present invention relates to a dying agent using natural henna, and more specifically, to a dying agent using natural henna with an increased absorption of dye and nutrients and resultant shortened dying time and with minimized damage to hair or scalp upon dying.

### DISCUSSION OF RELATED ART

Generally, the type of hair differs race to race, and as aged, the hair becomes gray.

Recently, black, gold, or other various colors are used for hair dying, not only for hiding gray hair but also for beauty.

However, existing dying agents which are basically made from chemicals may severely damage hair upon hair dying and some contain toxic substances which may injure skin and vision.

Korean Patent No. 10-0917164 titled “composition for hair dying agent” discloses a dying agent for preventing hair damage. However, it still uses chemicals or other synthetic substances. Thus, more and more users are interested in natural dyes, and thus, a dying method using henna attracts attention.

However, such dying methods using henna takes about two to about ten times longer for dying as compared with when synthetic dying agents are used, and this causes folks unwilling to use it.

### SUMMARY

To address the above issues, the present invention aims to provide a dying agent using natural henna with a greatly shortened dying time.

Another object of the present invention is to provide a dying agent using natural henna that does not damage hair or scalp upon dying.

To achieve the above objects, according to the present invention, a dying agent using natural henna comprises a fermented mixture of 80 wt % to 99 wt % of natural henna and 1 wt % to 20 wt % of fermenting agent and a natural additive mixed with the fermented mixture.

The fermented mixture is prepared by fermentation at 20° C. to 65° C. for 24 to 48 hours.

The fermenting agent is prepared by fermenting any one or more of sunflower roots, goat willow leaves, pyrethrum flower, prickly pear cactus, saururus chinensis, houttuynia cordata, eleutherococcus senticosus, and Japanese apricots.

The fermenting agent is an agrocybe cylindracea extract enzyme (AaeAPO).

The natural additive is any one or more of a natural color, an adsorptive mineral, a hair reinforcing agent, and a natural surfactant.

The natural color is any one or more of charcoal (black), ink (black), flavonoid (yellow), anthocyan (red), carthamin (red), rutin (yellow), quercetin (brown), tannin (red brown), chlorophyll (green), carotinoid (orange), crocin (orange), quinones (yellow, light red), shikonine (violet), alizarin (red), red cabbage (red, red violet), paprika (red, orange), red yeast rice (red), persicaria tinctoria (dark blue), flavin (yellow), gardenia (reddish), Schisandra chinensis (red), indigo (indigo blue), Chinese galls (purplish brown sappanwood (red), rosewood (red), logwood (red), madder (reddish yellow), and turmeric (yellow), and the natural color of 1 wt % to 15 wt % is added relative to the fermented mixture of 100 wt %.

The adsorptive mineral is any one or more of bentonite, zeolite, and tourmaline, and the adsorptive mineral of 5 wt % to 15 wt % is added relative to the fermented mixture of 100 wt %.

The hair reinforcing agent is any one or more of green tea, coffee powder, colostrt oysters, eggs, nuts, beans, gingko, royal jelly, astragalus membranaceus, goji berries, polygonum multillorum roots, camellia japonica linne, ginseng, evening primrose, American false daisy, perilla frutescens, and aloe vera, and the hair reinforcing agent of 3 wt % to 5 wt % is added relative to the fermented mixture of 100 wt %.

The natural surfactant is any one or more of coca-betaine, lecithin, saponin, and sodium cocoyl isethionate, and the natural surfactant of 0.5 wt % to 2 wt % is added relative to the fermented mixture of 100 wt %.

As described above, the dying agent using natural henna according to the present invention may significantly reduce dying time.

Further, the dying agent using natural henna according to the present invention may prevent damage to hair or scalp upon hair dying.

### BRIEF DESCRIPTION OF THE DRAWINGS

FIG. 1 is a view briefly illustrating a method for manufacturing a dying agent using natural henna according to the present invention.

FIG. 2 is a view illustrating a mixer of a dying agent using natural henna according to the present invention.

FIG. 3 is a view illustrating a wrapping container for wrapping up a dying agent using natural henna according to the present invention.

FIG. 4 is a flowchart illustrating a dying method using a dying agent using natural henna according to the present invention.

### DETAILED DESCRIPTION OF EMBODIMENTS

Specific features and advantages of the present invention are described below in detail with reference to the accompanying drawings. When determined to make the gist of the present invention unclear, the detailed description of functions and configurations of the present invention is omitted.

The present invention relates to a dying agent using natural henna, and more specifically, to a dying agent using natural henna with increased absorption of dye and nutrients and resultant shortened dying time and with minimized damage to hair or scalp upon dying.

Hereinafter, preferred embodiments of the present invention are described in detail with reference to the accompanying drawings.

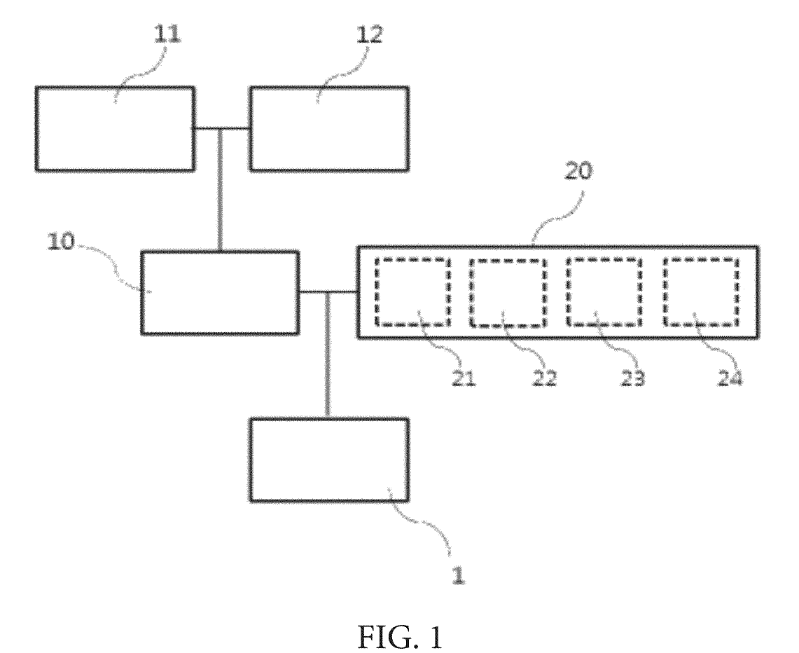

FIG. 1 is a view briefly illustrating a method for manufacturing a dying agent using natural henna according to the present invention.

According to the present invention, the dying agent 1 using natural henna is prepared by mixing a fermented mixture 10 with a natural additive 20, where the fermented mixture 10 is prepared by fermenting of a mixture of 80 wt % to 99 wt % of natural henna 11 and 1 wt % to 20 wt % of fermenting agent 12.

The natural henna 11 grows wild primarily in hot and dry regions, such as Egypt, India, or Arab countries, and is a lythraceous plant called lawsonia inermis.

A main element of henna is lawsone, 2-hydroxy 1,4-naphathoquinone and this substance reacts to color hair, nails, fingers, palms, and soles.

Further, natural henna has the nature of bonding protein and thus recovers damaged keratin of hair to make the hair glossy and healthy.

Further, natural henna, as compared with other types of dye, is less irritative and toxic enough to be available for the pregnant, and reduce scalp fever and removes itch and dandruff.

Moreover, natural henna may supply inorganic substances or other nutrients to make hair thicker and preventing losing hair. Natural henna also works for treatment of losing hair.

Whereas chemical synthetic dying agents may permanently dye hair while destroying and damaging the melanin pigment and tissues in the hair, natural henna 11 smoothly sticks and wraps around the hair to coat and nourishes, treats and protects the hair.

The natural henna may come in powder or sticky liquid.

The natural henna, when coming in liquid, may be rendered to have viscosity by mixing natural henna with purified water in a predetermined ratio (natural henna:purified water=1 to 2:3). The mixture of natural henna and purified water, when left at room temperature for a long time, may experience oxidation to deteriorate the capacity of dying. Thus, it is preferable to use it within 48 hours, preferably within 24 hours.

The fermenting agent 12 is added to naturally remove toxicity that may remain in the natural substances or elements of the dying agent according to the present invention and to reinforce the dying and coloring capacity.

The fermenting agent 12 is characterized to be prepared by fermenting one or more of sunflower roots, goat willow leaves, pyrethrum flower, prickly pear cactus, saururus chinensis, houttuynia cordata, eleutherococcus senticosus, and Japanese apricots.

The fermenting agent has excellent anti-oxidative and anti-inflammatory activities and well decomposes and flushes toxins out. Thus, the fermenting agent may remove and mitigate the toxins that may remain in the natural substances added to the dying agent according to the present invention or may be created by organic bonding among the natural substances.

Further, the fermenting agent may minimize scalp inflammation that may occur during the course of dying and may drive out heavy metals and contaminants that accumulate on the scalp and hair.

Sunflower roots and goat willow leaves are effective in removing heavy metals (e.g., cadmium or zinc) and are high resistant to toxins and well decomposes contaminants.

Pyrethrum flower has pyrethrin and cinerin as its major elements and presents insecticidal and toxin decomposing effects without harming the human body.

Prickly pear cactus contains phyto gelatin with anti-oxidation and so detoxifies and flushes out heavy metals. saururus chinensis and houttuynia cordata have anti-inflammatory properties due to actions of quercetin and quercitrin.

Acanthosides B and D of eleutherococcus senticosus and picric acid of Japanese apricots decompose toxins and protect scalp.

Although not limited to a particular fermenting method, it is preferably mixed and fermented together with sugar and EM fermented broth for about one to about six months to lead to activation of effective elements and enhanced anti-oxidation effects.

The fermenting agent is characterized to be an agrocybe cylindracea extract enzyme (AaeAPO).

AaeAPO (agrocybe cylindracea extract enzyme) classified as an aromatic peroxygenase (APO) is a sort of eumycetes enzyme and does not lose activities nor is it destroyed even when remaining isolated, and it is very excellent in stability and decomposes toxins.

In particular, it shows an excellent capacity of removing toxins by decomposing methylimidazole which is a carcinogenic aromatic compound.

The fermenting mixture 10 is characterized to be prepared by mixing 80 wt % to 99 wt % of natural henna with 1 wt % to 20 wt % of fermenting agent and to ferment the mixture at 20° C. to 65° C. for 24 to 48 hours.

The fermenting agent shows a weak effect when contained in less than 1 wt %, and when contained in more than 20 wt %, the content of natural henna added is relatively reduced, leading to a deteriorated dying capacity of the dying agent.

The temperature of fermentation is preferably 20° C. to 65° C. When the temperature is less than 20° C. the fermentation does not go well, and when the temperature is more than 65° C., the enzyme may be destroyed. Thus, it is preferable not to depart from the temperature range.

Meanwhile, if the time of fermentation is less than 24 hours, it fails to bring up with a satisfactory result, and if the time exceeds 48 hours, the enzyme may be destroyed, leading to rot and odors.

As the fermentation proceeds, it may let the hair glossier and smooth for later dying and nourish the hair to further thicken the hair (preventing losing hair by nourishment).

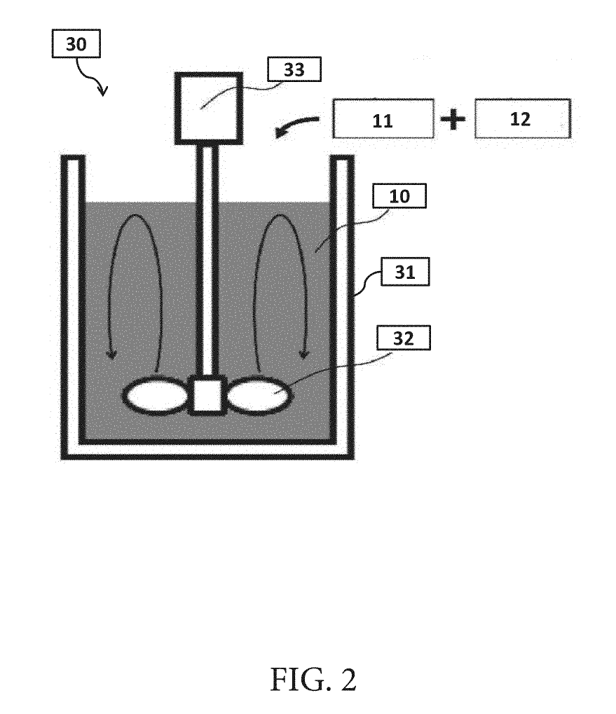

In order to more evenly mix the natural henna 11 and the fermenting agent 12, a mixer may come in use. FIG. 2 shows a mixer 30 for mixing and preparing a dying agent using natural henna according to the present invention.

The mixer 30 preferably includes a tank 31 to contain substances (natural henna and fermenting agent) to be mixed, an impeller 32 for mixing the substances, and a motor 33 for rotating the impeller. The motor 33 is linked to the impeller 32 and spins to evenly mix the natural henna 11 and the fermenting agent 12.

Further, the impeller 32 may be generally shaped as a propeller or may have other various shapes such as a cylinder or plate without changing the function of evenly mixing the natural henna 11 and the fermenting agent 12.

The mixer 30 is not limited to the purpose of mixing the natural henna and the fermenting agent and may also be used to mix the prepared fermented mixture 10 with a natural additive 20.

If only natural henna is used as a dye, it fails to produce various colors, rather with limited colors such as brown or red brown and a limited coloring capacity. Accordingly, the natural additive 20 adds to enhance the dying properties (dying time, coloring capacity) and to reinforce the nutrients to be supplied to the hair.

The natural additive is characterized to be one or more of a natural color 21, an adsorptive mineral 22, a hair reinforcing agent 23, and a natural surfactant 24.

The natural color 21 adds to adjust the coloring capacity and feeling of color of the dying agent and is any one or more of charcoal (black), ink (black), flavonoid (yellow), anthocyan (red), carthamin (red), rutin (yellow), quercetin (brown), tannin (red brown), chlorophyll (green), carotinoid (orange), crocin (orange), quinones (yellow, light red), shikonine (violet), alizarin (red), red cabbage (red, red violet), paprika (red, orange), red yeast rice (red), persicaria tinctoria (dark blue), flavin (yellow), gardenia (reddish), Schisandra chinensis (red), indigo (indigo blue), Chinese galls (purplish brown), sappanwood (red), rosewood (red), logwood (red), madder (reddish yellow), and turmeric (yellow).

For example, when desiring to dye the hair in black, a predetermined amount of charcoal or ink adds to the fermented mixture. The concentration of color dyed varies depending on the amount of charcoal added. That is, the more charcoal is added, the darker the hair becomes.

Further, in case persicaria tinctoria is used as dye, its leaves are dried in shade and is ground in a powder of 2000 mesh, and a predetermined amount thereof is added. The concentration of color dyed varies depending on the amount of persicaria tinctoria added.

Further, when persicaria tinctoria (or its leaves) is used as a natural dye, it may neutralize the toxins thanks its detoxifying properties. It also contains anti-cancer substances.

Further, substances (colors) added to the dying agent using natural henna to adjust the color of hair according to the present invention are not limited as long as the substances are natural substances (colors), not synthetic colors, and for example, walnuts, chamomile or other natural substances (colors) may also be used.

Further, the color of hair to be dyed (mixed color obtained by mixing two or more kinds of natural substances) may be adjusted in various manners by mixing several types of natural substances, not alone a single type of natural substance.

For example, a natural substance which is blue and another natural substance which is yellow may be mixed together in a predetermined ratio to produce green.

More specifically, animal natural color elements include hemocyanin, bile pigment, pterin, carotinoid, quinones, and cochineal, vegetable natural color elements include chlorophyl, carotinoid, anthocyan, flavonoid, quinones, and gardenia, and microorganism color elements include chlorella, spirulina, and red yeast rice.

Further, the process of preparing the natural color includes extracting from source materials, filtering, concentrating, refining, sterilizing, color adjusting, liquidizing, or powdering. The natural color benefits higher stability and reliability, better coloring, easier preparing, and eco-friendliness as compared with artificial synthetic colors.

1 wt % to 15 wt % of natural color is added relative to 100 wt % of fermented mixture. If less than 1 wt % of natural color is added, it is less effective, and if more than 15 wt % of natural color is added, it is difficult to control the feeling of color given the chemical characteristics (oxidative property) of each element. Thus, it is preferable not to depart from the range.

The adsorptive mineral 22 is for building up an environment where the dye elements or nutrients of the dying agent using natural henna may be absorbed well and may include any one or more of bentonite, zeolite, and tourmaline.

The adsorptive mineral has high cation exchangeability, absorptivity, and swellability, and more specifically, the adsorptive mineral contains 66 kinds of natural minerals, such as silicon dioxide, aluminum oxide, magnesium oxide, iron oxide, calcium oxide, calcium hydroxide, and sodium oxide, which emit far infrared radiations and anions that mutually react to help absorption of the dye elements (natural henna and natural color) and other nutrients.

In other words, conventional dying using natural henna takes a long dying time, but according to the present invention, adding the adsorptive mineral may shorten the dying time and assist in absorption of other nutrients, leading to reinforced hair protection.

Further, it may adsorb various harmful substances including wastes, sebum, and heavy metals to keep the hair clean and elastic while nourishing the hair.

5 wt % to 15 wt % of adsorptive mineral is added relative to 100 wt % of fermented mixture. If it is added in less than 5 wt %, it is less effective, and if added in more than 15 wt %, such increase in content does not play a significant role and rather deteriorates the mixibility with other substances (fermented mixture, natural additive) to render it difficult to apply upon dying. Thus, it is preferable not to depart from the range.

The hair reinforcing agent 23 is added to minimize side effects such as losing hair due to irritating scalp and damage to the cuticle layer of hair that may arise upon dying. The hair reinforcing agent 23 may be any one or more of green tea, coffee powder, colostrum, oysters, eggs, nuts, beans, gingko, royal jelly, astragalus membranaceus, goji berries, polygonum multiflorum roots, camellia japonica linne, ginseng, evening primrose, American false daisy, perilla frutescens, and aloe vera.

Catechin of green tea suppresses generation of dihydrotestosterone (DHT) and other elements of green tea, e.g., tannin, shrink skin pores to prevent hair loss.

Caffeine in the coffee powder prompts intracellular metabolism to properly nourish hair roots and functions as an energy source for hair to retard hair loss.

Oysters are rich in zinc which prompts metabolism in cells and tissues and gets rid of active oxygen that may negatively affect hair. Further, zinc is involved in synthesis of collagen to prevent hair from thinning and aging.

Lactoferrin in colostrum is synthesized together with proteins, recovers damaged hair, removes active oxygen with strong antioxidants, and increases scalp immunity.

Eggs and nuts are rich in vitamin B3, also called niacin or nicotinic acid, which thickens hair or prompts hair growth. Eggs and nuts are also rich in biotin which mitigates scalp inflammation and prevents hair from falling out.

astragalus membranaceus, goji berries, polygonum multiflorum roots, camellia japonica linne, and ginseng are rich in saponin and prompts blood circulation and resultantly hair growth.

Bins, gingko, and royal jelly are rich in flavonoid which cleanses the scalp, and retards the propagation of dandruff, germs, and demodex mites to prevent hair loss, or as a strong antioxidant, prevents the scalp and hair from aging.

aloe vera, perilla frutescens, and American false daisy reinforce the protection film of scalp to prevent hair from falling out.

evening primrose suppresses sebum secretion from the scalp, well nourishes the scalp, and increases moisturizing.

3 wt % to 5 wt % of the hair reinforcing agent is added relative to 100 wt % of fermented mixture. If the hair reinforcing agent is added in less than 3 wt %, it does not exhibit a significant effect, and if the hair reinforcing agent is added in more than 5 wt %, the viscosity increases, rendering it difficult to mix with other elements of the dying agent. Thus, it is preferable not to depart from the range.

The natural surfactant 24 allows the elements (natural henna, fermenting agent, and natural additive) of the dying agent using natural henna to mix well together so that the elements may organically bond together. The natural surfactant 24 may be used as an agent for allowing the dying agent viscosity.

The natural surfactant is characterized to be any one or more of coco-betaine, lecithin, saponin, and sodium cocoyl isethionate.

The natural surfactant is extracted from natural materials, such as coconuts, soybeans, or eggs and it is harmless to skin and excellent in skin miniaturization.

0.5 wt % to 2 wt % of natural surfactant is added relative to 100 wt % of fermented mixture. If the natural surfactant is added in less than 0.5 wt %, it does not exhibit a significant effect, and if it is added in more than 2 wt %, excessive surface activity occurs, resulting in the dying agent penetrating into the hair. Thus, it is preferable not to depart from the range.

Further, the dying agent according to the present invention may further include a natural acid as a natural additive to adjust pH.

The dying agent using natural henna according to the present invention may exhibit a highest dying effect when its pH remains in a range from 4 to 6. Any acid powder, such as citric acid, tartaric acid, adipic acid, DL-malic acid, and lactic acid, may be used to adjust the coloring capacity for dying the hair.

The dying agent using natural henna according to the present invention may be formed in a powder or cream type with a predetermined viscosity or may also be formed in a liquid type.

This is varied by adding a natural additive or purified water to the dying agent using natural henna. For example, purified water may be added to prepare a liquid-type dying agent or a natural oil may be added to prepare a cream-type dying agent.

It may also be varied depending on the amount of purified water or natural oil added. As the amount of purified water or natural oil added increases, the dying agent would turn from powder type through cream type to liquid type.

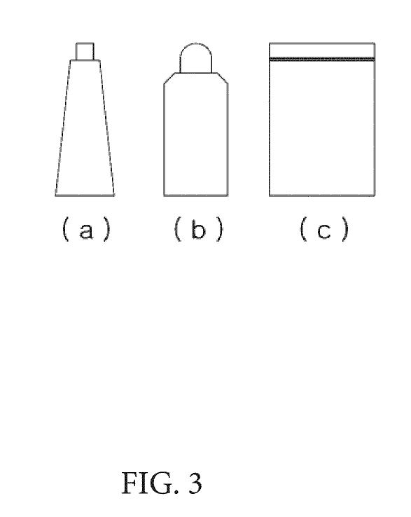

As set forth above, in case the dying agent is prepared in a cream type, it is preferably contained in a tube-type wrapping material as shown in FIG. 3(a), so as to be squeezed out in a predetermined quantity when used. In case the dying agent is prepared in a liquid type, it may be contained in a bottle or can as shown in FIG. 3(b). In case the dying agent is prepared in a powder type, it may be wrapped in a wrapping material, such as a plastic or paper pouch as shown in FIG. 3(c).

That is, the dying agent using natural henna according to the present invention may be contained in any one of a tube, can, bottle, and a pouch.

Further, the dying agent using natural henna prepared in a cream, liquid, or powder type as described above, when prepared in a cream type, may be mixed with natural oil or purified water to have a viscosity of 1,000 cP to 50,000 cP. When the dying agent is prepared in a liquid type, it may be mixed with natural oil and purified water to have a viscosity of 1 cP to 999 cP.

Further, when the dying agent is prepared in a powder type, it may be formed in a powder with a grain size of 100 mesh to 2,000 mesh.

Further, the dying agent may be produced, e.g., in the form of a massage pack and it may be used for scalp and facial detoxifying massage.

Further, the dying agent may add a fatty acid and sodium hydroxide to be formed in a solid and may be used as a soap. When produced as a soap, the natural henna and natural substances may protect and cleanse well scalp and skin.

Further, the dying agent may add a shampoo element, formed in a shampoo type that enables both cleansing and shampooing. The shampoo element includes a surfactant, a viscosity increasing agent, or a conditioning agent.

Accordingly, it provides such effects as cleansing, treatment, and dying.

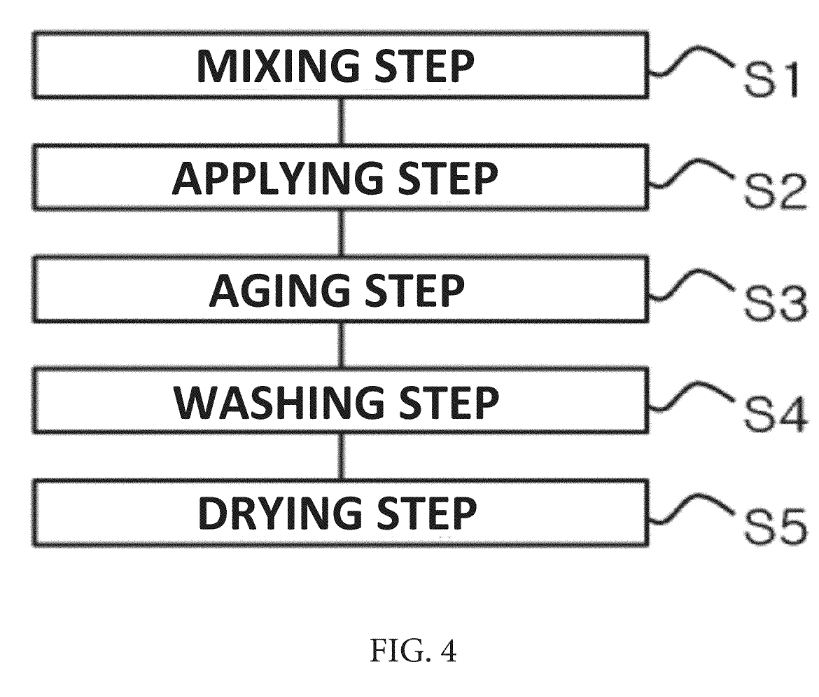

FIG. 4 illustrates a dying method using a dying agent using natural henna according to the present invention. The method includes the step S1 of mixing substances for dying, the step S2 of applying the mixture to hair to be dyed, the step S3 of aging the substances applied to the hair, the step S4 of washing out the substances which have been applied to the hair and aged, and the step S5 of drying the washed hair.

More specifically, in the mixing step S1, the dying agent natural henna, which is in a powder type, and water are mixed in a ratio of 1:1 to 1:5. Accordingly, the dying agent using natural henna is mixed in a form with a predetermined viscosity.

Further, in case the dying agent using natural henna is in a cream or liquid type, the mixing step may be omitted.

As described above, if the dying agent using natural henna is mixed in the form with a predetermined viscosity, may be applied to the dry hair in the form with the viscosity by the applying step S2.

Further, if the dying agent using natural henna is evenly applied to the hair in the applying step S2, the hair is covered with a plastic film or wrap in the aging step S3 to cut off influx of air into the hair and to evenly dye the hair.

In a predetermined time, the washing step S4 is performed to wash out the dying agent. After the washing step S4, the drying step S5 is performed to dry the hair using dry air with a predetermined temperature.

In the drying step S5, the dry air with a minimum temperature of 40° C. is used to evenly dye and color the hair.

The dying agent using natural henna according to the present invention adopts natural substances and thus is less irritatative and eco-friendlier as compared with synthetic dying agents. The washed-out waste is also harmless to the human body.

Further, the eco-friendly natural substances may provide nourishment, elasticity, and shine to hair and scalp to add moisture and thicken hair, resultantly preventing hair loss.

While the present invention has been shown and described with reference to exemplary embodiments thereof, it will be apparent to those of ordinary skill in the art that various changes in form and detail may be made thereto without departing from the spirit and scope of the present invention as defined by the following claims.

What is claimed is:

1. A dying agent using natural henna, the dying agent comprising a fermented mixture of 80 wt % to 99 wt % of natural henna and 1 wt % to 20 wt % of fermenting agent and a natural additive mixed with the fermented mixture.

2. The dying agent of claim 1, wherein the fermented mixture is prepared by fermentation at 20° C. to 65° C. for 24 to 48 hours.

3. The dying agent of claim 1, wherein the fermenting agent is prepared by fermenting any one or more of sunflower roots, goat willow leaves, pyrethrum flower, prickly pear cactus, saururus chinensis, houttuynia cordata, eleutherococcus senticosus, and Japanese apricots.

4. The dying agent of claim 1, wherein the fermenting agent is an agrocybe cylindracea extract enzyme (AaeAPO).

5. The dying agent of claim 1, wherein the natural additive is any one or more of a natural color, an adsorptive mineral, a hair reinforcing agent, and a natural surfactant.

6. The dying agent of claim 5, wherein the natural color is any one or more of charcoal (black), ink (black), flavonoid (yellow), anthocyan (red), carthamin (red), rutin (yellow), quercetin (brown), tannin (red brown), chlorophyll (green), carotinoid (orange), crocin (orange), quinones (yellow, light red), shikonine (violet), alizarin (red), red cabbage (red, red violet), paprika (red, orange), red yeast rice (red), persicaria tinctoria (dark blue), flavin (yellow), gardenia (reddish), Schisandra chinensis (red), indigo (indigo blue), Chinese galls (purplish brown), sappanwood (red), rosewood (red), logwood (red), madder (reddish yellow), and turmeric (yellow), and wherein the natural color of 1 wt % to 15 wt % is added relative to the fermented mixture of 100 wt %.

7. The dying agent of claim 5, wherein the adsorptive mineral is any one or more of bentonite, zeolite, and tourmaline, and wherein the adsorptive mineral of 5 wt % to 15 wt % is added relative to the fermented mixture of 100 wt %.

8. The dying agent of claim 5, wherein the hair reinforcing agent is any one or more of green tea, coffee powder, colostrum, oysters, eggs, nuts, beans, gingko, royal jelly, astragalus membranaceus, goji berries, polygonum multiflorum roots, camellia japonica Billie, ginseng, evening primrose, American false daisy, perilla fruteseens, and aloe vera, and wherein the hair reinforcing agent of 3 wt % to 5 wt % is added relative to the fermented mixture of 100 wt %.

9. The dying agent of claim 5, wherein the natural surfactant is any one or more of coco-betaine, lecithin, saponin, and sodium cocoyl isethionate, and wherein the natural surfactant of 0.5 wt % to 2 wt % is added relative to the fermented mixture of 100 wt %.
